(19) United States
(12) Patent Application Publication
(10) Pub. No.: US 2017/0246102 A1
(43) Pub. Date: Aug. 31, 2017

(54) Hair Products Containing Branched Amine Functionalized Silicone Compounds

(71) Applicant: Colgate-Palmolive Company, New York, NY (US)

(72) Inventors: Ramon Arnoldo Lopez Baca, Del.Miguel Hidalgo (MX); Yumiko Raquel GARCIA MACIAS, Del.Miguel Hidalgo (MX)

(73) Assignee: Colgate-Palmolive Company, New York, NY (US)

(21) Appl. No.: 15/504,667

(22) PCT Filed: Aug. 18, 2014
(86) PCT No.: PCT/US2014/051460

§ 371 Date: Feb. 17, 2017

(51) Int. Cl. A61K 8/898; A61K 8/34; A61Q 5/12

(52) U.S. Cl. CPC A61K 8/898; A61Q 5/12; A61K 8/342

(57) ABSTRACT

The present disclosure pertains to compositions that include branched amine functionalized silicone compounds, methods involving the application of such compounds to hair, methods for making hair conditioners using such compounds, and uses of compositions containing such compounds for conditioning of hair for one or both of reducing tangling and increasing the ease of combing.

### BACKGROUND

There presently exist a wide variety of consumer products for cleansing and conditioning of hair. Hair conditioners include leave-on and rinse-off products. Shampoos may consist of dedicated products that are solely for cleansing, or may include hair conditioning agents that permit a single product to both cleanse and condition the hair. Other products that are not intended solely as cleansing or conditioning compositions may include conditioning agents. The active components of conditioning products may be included to enhance the softness, manageability, luster, or moisture content of hair, to reduce dryness, tangling, brittleness, or color loss, or to provide any combination of these benefits. While hair conditioners initially consisted of naturally-occurring oils, modern hair care products can make use of silicone, fatty alcohols, quaternary ammonium compounds, and other actives in order to accomplish a conditioning effect.

Many existing conditioning products may be effective to provide one or several particular benefits, but are unable to confer another desirable effect. Accordingly, there exists an ongoing need for conditioning agents and products that are made from such agents that provide any of the traditionally desired benefits of hair conditioning.

### BRIEF SUMMARY

The present disclosure concerns methods comprising applying to the hair of a mammal a composition that includes a compound having the formula (I)

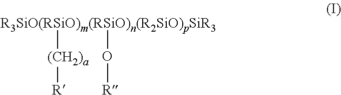

wherein
R is a C₁-C₄ alkyl group;
R′ is an amine or salt thereof;
R″ is (R₂SiO)ₓ or (R₂SiO)_{y}(RSiO)_{w}[(CH₂)₃R′]_{z};
a is 1-10;
m is 1-5;
n is 3-20;
p is 300-500;
x is 50-200;
y is 20-100;
w is 0-10;
z is 0-5; and,
x+y+w+z+p=500-700.

Also disclosed are compositions for the conditioning of hair, methods for making a hair conditioner, and uses of a composition for conditioning of hair_{;} each of which involve a compound according to the above-described formula (I).

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Provided herein are methods comprising applying to the hair of a mammal a composition that includes a compound having the formula (I)

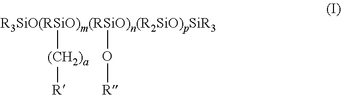

wherein

R is a C₁-C₄ alkyl group;

R′ is an amine or salt thereof;

R″ is (R₂SiO)ₓ or (R₂SiO)_{y}(RSiO_{w}[(CH₂)₃R′]_{z};

a is 1-10;

m is 1-5;

n is 3-20;

p is 300-500;

x is 50-200;

y is 20-100;

w is 0-10;

z is 0-5; and,

x+y+w+Z+p=500-700.

Emulsion SR2 silicone, which is available from Provista S.A. de CV of Mexico, represents an exemplary composition that includes compound according to formula (I).

The compound according to formula (I) may have a weight average molecular weight of 1,000 to 200,000. For example, the weight average molecular weight of the compound of formula (I) may be 2,000 to 150,000, 5,000 to 120,000, 10,000 to 100,000, 20,000 to 75.000, 38,000 to 52,000, or 40,000 to 48,000. In certain embodiments, the molecular weight is about 45,000.

In some embodiments, the compound of formula (I) is not a film-forming polymer.

With respect to the compound of formula (1), at least 20%, at least 40%, at least 50%, at least 50%, at least 70%, at least 80%, at least 85%, at least 90%, or at least 95% of the R groups in the (RSiO) or (R₂SiO) moieties are methyl.

R′ of formula (I) may be, for example, —NH₂, NHR, —N(R)₂, —NH—(CH₂)_{b}—NH₂, or —N(R)₃⁻, wherein b is 1 to 6. In certain instances, b may be 1-5, 1-4, 1-3, 1-2, or 2.

The compound having the formula (I) may be present in an amount of 0.01 to 5.0% by weight of the composition of the presently disclosed methods. For example, the compound may be present in an amount of 0.05 to 3.0% by weight, 0.1 to 2,0% by weight, 0.2 to 1.0% by weight, or 0.25 to 0.5% by weight, or 0.3 to 0.4% by weight. In some embodiments, the compound having the formula (I) is present in an amount of about 0.2% by weight, about 0.25% by weight, about 0.3% by weight, about 0.35% by weight, about 0.4% by weight, about 0.5% by weight, about 0.6% by weight, about 0.7% by weight, about 0.8% by weight, about 0.9% by, weight, or about 1% by weight.

The compositions of the present methods may further comprise one or more of a thickener, a viscosity enhancer, a pH modifier, a surfactant, an emulsifier, an oil, a detangling agent, a moisturizer, a hair shine enhancer, a hair strength enhancer, a preservative, and fragrance, For example, the compositions may comprise each of a thickener, a viscosity enhancer, a pH modifier, a surfactant, an emulsifier, an oil, a detangling agent, a moisturizer, a hair shine enhancer, a hair strength enhancer, a preservative, and fragrance, In a certain embodiment, the composition comprises water, hydrophobically modified hydroxyethylcellulose, cetyitrimethyammonium chloride, cetyl stearyl alcohol, glyceryl monostearate, mineral oil, a cationic silicone emulsion, honey, hydrolyzed silk, a preservative, fragrance, NaOH, and citric acid. The compositions may take the form of a hair conditioner, such as a rinse-off hair conditioner.

The compositions of the present methods may comprise fatty alcohols, such as stearyl alcohol, cetyl alcohol, or both. For example, the compositions may include cetostearyl alcohol. The compositions may also or alternatively include silicones, such as dimethicone, dimethiconol, cyclopentasiloxane, an amodiniethicone, or any combination thereof. The compositions may also or alternatively include cationics, including surfactants like cetrimonium chloride or quaternium 82, or polymeric cationics, such as the polyquaternium series (e.g., polyquaternium-6, -7, or -8) or cationic guar gum. The compositions may further comprise oils, such as mineral oil. Further examples of fatty alcohols, silicones, cationics, and oils are well known among those skilled in formulating hair products.

The presently disclosed methods may further comprise detangling the hair after application of the composition. Detangling may be performed mechanically, such as with a comb or brush, or manually. Detangling may involve the application of a various peak load forces. The presently disclosed methods accord with the unexpected discovery that when a composition comprising a compound according to formula (I) is applied to hair, the peak load forces that are required to detangle the hair are lower than those that are required to detangle a comparable portion of hair to which the present compositions have not been applied, and are even lower than the forces that are required after application of alternative compositions, such as hair conditioners, that do not include a compound according to formula (I). This feature of the present methods and others are discussed more fully infra. The peak load force that is required to detangle a tress of hair weighing about 3.5 g after application of a composition according to the present claims may be, for example, no more than about 180 gmf, no more than about 160 gmf, no more than about 150 gmf, no more than about 140 gmf, no more than about 130 gmf, or no more than about 120 gmf.

The present disclosure also pertains to compositions for the conditioning of hair comprising a compound having the formula (I)

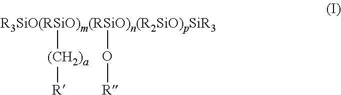

wherein

R is a C₁-C₄ alkyl group;

R′ is an amine or salt thereof;

R″ is (R₂SiO)ₓ or (R₂SiO)_{y}(RSiO)_{w}[(CH₂)₃R′]_{z};

a is 1-10;

m is 1-5;

n is 3-20;

p is 300-500;

x is 50-200;

y is 20-100;

w is 0-10;

z is 0-5; and,

x+y+w+z+p=500-700;

and, one or more further ingredients that are suitable for inclusion in the composition for use on the hair of a mammal. For example, the present compositions may be a hair conditioner, such as a rinse-off hair conditioner.

Unless otherwise specified, any component, element, attribute, or step that is disclosed with respect to one embodiment of the present methods, compositions, and uses may apply to any other embodiment that is disclosed herein. For example, the presently disclosed compositions may have any of the characteristics of the composition that are used pursuant to the present methods, and vice versa, and the compounds that are used in the presently disclosed methods may have any of the characteristics of the compounds of the presently disclosed compositions, and vice versa.

Thus, the compound of formula (I) in the present compositions may have any of the characteristics of the compounds described above with respect to the disclosed methods that involve the use of a compound according to formula (I). In addition, the present compositions may have any of the characteristics of the compositions that are used pursuant to the above-described methods.

As used herein, one or more further ingredients that are suitable for inclusion in the composition for use on the hair of a mammal refers to any ingredient that is characteristic of a hair product, such as a hair conditioner. The present compositions may also include components that are not necessarily characteristic of a hair product, but must include at least one component that is characteristic of a hair product. Exemplary ingredients that are characteristic of a hair product include fatty alcohols, silicones, oils, and cationics. Examples of these ingredients are described above in connection with the present methods, and further examples of which are well known among those skilled in formulating hair products. The component that is characteristic of a hair product may be, but is not necessarily, an ingredient that is not typically included in a non-hair product, such as in a fabric conditioner.

The present disclosure also pertains to methods for making a hair conditioner comprising preparing a composition that includes a compound having the formula (I), as previously described, and one or more further ingredients that are suitable for inclusion in the composition for use on the hair of a mammal. The compound of formula (I) in the present methods may have any of the characteristics of the compounds described above with respect to the above described methods that involve the use of a compound according to formula (I). In addition, the compositions of the present methods may have any of the characteristics of the compositions that are used pursuant to the above-described methods. Likewise, the one or more further ingredients that are suitable for inclusion in the composition for use on the hair of a mammal may have any of the characteristics that are described in connection with the above-described compositions, such as in the immediately preceding paragraph.

Also disclosed herein are uses of a composition for conditioning of hair for one or both of reducing tangling of the hair and increasing ease of combing of the hair, wherein the composition comprises a compound having the formula (I), as previously described. For example, the composition of the presently disclosed uses may be a hair conditioner, such as a rinse-off hair conditioner. The composition may further include one or more further ingredients that are suitable for inclusion in the composition for use on the hair of a mammal. The compound of formula (I) in the present uses may have any of the characteristics of the compounds described above with respect to the above described methods that involve the use of a compound according to formula (I). In addition, the compositions of the present uses may have any of the characteristics of the compositions that are used pursuant to the above-described methods. Likewise, the one or more further ingredients that are suitable for inclusion in the composition for use on the hair of a mammal may have any of the characteristics that are described in connection with the above-described compositions.

### EXAMPLES

### Example 1

Clean, bleached hair was prepared into 3.5±0.1 g tresses and pretreated with a cleansing solution of 20% ammonium lauryl sulfate then dried overnight.

Hair tresses were wet under tap water at 25° C. and then treated with 0.5 mL of a rinse-off conditioner prototype (see Table 1, below) containing a composition that includes a compound according to formula (I), or a comparison active, each at 0.35% by weight of the hair conditioner. Hair was rubbed for one minute with the test conditioner, and then rinsed again under tap water for one further minute. A Dia-Stroh instrument (Broomall, Pa.) was used to determine the peak load force (gmf), which indicates the force required to untangle a hair tress. The instrument also measured the total work force (joules) as the force necessary to comb a hair tress.

The rinse-off conditioner formula is listed in Table 1 below, and included 0.35% by weight of a composition that included a compound having the formula (I) or 0.35% by weight of Silsoft HC400 amino bispropyl dimethicone (used for comparative purposes).

Table 1, below, lists the components of the prototype conditioner formula used, including the test composition present at 0.35% by weight and the other materials based on active amount.

TABLE 1

| Component | Weight % | Weight % |
|---|---|---|
| Water and minors | Q.S. | Q.S. |
| (fragrance, pH agent, preservative, etc.) | | |
| Stearyl Alcohol | 3.15 | 3.15 |
| Cetyl Alcohol | 1.35 | 1.35 |
| Cetrimonium Chloride | 1 | 1 |
| Mineral Oil | 0.5 | 0.5 |
| Glyceryl Stearate | 0.45 | 0.45 |
| Silsoft HC400 Amino bispropyl dimethicone | 0.35 | 0 |
| Composition comprising a compound | 0 | 0.35* |
| having the formula (I) | | |

*The active compound was included in the test formulation in an amount of 0.30% by weight, along with 0.05% by weight of hexadecyl trimethyl ammonium chloride, which positively affects the performance of the active compound

Five separate tresses were measured for each composition under test. Each tress was measured three times, tier a total of 15 readings for each test composition.

Table 2 shows the average force (gmf) required to detangle a hair tress and the average force (joules) required to comb a hair tress:

TABLE 2

| | Average force (gmf) | Average Force (joules) |
|---|---|---|
| Composition | to detangle | to comb |
| Comparative | 186.64 | 0.2148 |
| Inventive | 118.97 | 0.1757 |

The test composition containing a compound according to formula (I) successfully reduced the force necessary to untangle hair, and the force necessary to comb hair, relative to the comparative composition.

What is claimed is:

1. A method comprising:
applying to the hair of a mammal a composition that includes a compound having the formula (I) 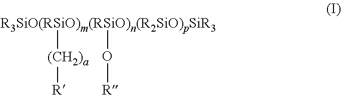
wherein
is a C₁-C₄ alkyl group;
R′ is an amine or salt thereof;
R″ is (R₂SiO)ₓ or (R₂SiO)_{y}(RSiO)_{w}[(CH₂)₃R′]_{z};
a is 1-10;
m is 1-5;
n is 3-20;
p is 300-500;
x is 50-200;
y is 20-100;
w is 0-10;
z is 0-5; and,
x+y+w+z+p=500-700.

2. The method of claim 1, wherein the compound of formula (I) has a weight average molecular weight of 1,000 to 200,000.

3. (canceled)

4. (canceled)

5. The method of claim 1, wherein the compound of formula (I) has a weight average molecular weight of about 45,000.

6. The method of claim 1, wherein at least 80% of the R groups in the (RSiO) or (R₂SiO) moieties of the compound of formula (I) are methyl.

7. The method of claim 1, wherein R′ is —NH₂, NHR, —N(R)₂, —NH—(CH₂)_{b}—NH₂, or —N(R)₃⁺, and wherein b is 1 to 6.

8. The method of claim 1, wherein R″ is (R₂SiO)ₓ.

9. The method of claim 1, wherein the compound of formula (I) is present in the composition an amount of 0.05 to 3% by weight.

10. (canceled)

11. (canceled)

12. The method of claim 1, wherein the composition further comprises a fatty alcohol.

13. The method of claim 12, wherein the fatty alcohol comprises stearyl alcohol, cetyl alcohol, or a mixture of stearyl alcohol and cetyl alcohol.

14. The method of claim 1, further comprising detangling the hair.

15. (canceled)

16. (canceled)

17. (canceled)

18. A composition for conditioning hair, comprising:
a compound having the formula (I) 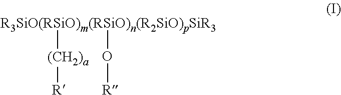
wherein
R is a C₁-C₄ alkyl group;
R′ is an amine or salt thereof;
R″ is (R₂SiO)ₓ or (R₂SiO)_{y}(RSiO)_{w}[(CH₂)₃R′]_{z};
a is 1-10;
m is 1-5;
n is 3-20;
p is 300-500;
x is 50-200;
y is 20-100;
w is 0-10;
z is 0-5; and,
x+y+w+z±p=500-700,
and
one or more further ingredients that are suitable for inclusion in the composition for use on the hair of a mammal.

19. The composition of claim 18, wherein the compound of formula (I) has a weight average molecular weight of 1,000 to 200,000.

20. (canceled)

21. (canceled)

22. (canceled)

23. The composition of claim 18, wherein at least 80% of the R groups in the (RSiO) or (R₂SiO) moieties of the compound of formula (I) are methyl.

24. The composition of claim 18, wherein R′is —NH₂, NHR, —N(R)₂, —NH—(CH₂)_{b}—NH₂, or —N(R)₃⁺, wherein b is 1 to 6.

25. The composition of claim 18, wherein R″ is (R₂SiO)ₓ.

26. The composition of claim 18, wherein the compound of formula (I) is present in the composition an amount of 0.05 to 3% by weight.

27. (canceled)

28. (canceled)

29. The composition of claim 18, wherein the further ingredient comprises a fatty alcohol.

30. The composition of claim 29, wherein the fatty alcohol comprises stearyl alcohol, cetyl alcohol, or a mixture of stearyl alcohol and cetyl alcohol.

31. The composition of claim 18, wherein the composition is a rinse-off hair conditioner.

32. A method for making the composition of claim 18 comprising mixing the compound having the formula (I) with the one or more further ingredients that are suitable for inclusion in the composition for use on the hair of the mammal.

33. (canceled)
